(19) United States
(12) Patent Application Publication
(10) Pub. No.: US 2016/0106874 A1
(43) Pub. Date: Apr. 21, 2016

(54) SYSTEMS AND METHODS TO CLEAN CHILDREN'S OBJECTS

(71) Applicant: TidyToyz, LLC, San Diego, CA (US)

(72) Inventor: Aurora I. Torres, San Diego, CA (US)

(21) Appl. No.: 14/515,410

(22) Filed: Oct. 15, 2014

(51) Int. Cl. A61L 2/18

(52) U.S. Cl. CPC A61L 2/18

(57) ABSTRACT

The systems, methods, platforms and devices described herein comprise: a first basket comprising at least one children's object and one or more holes configured to allow fluid to flow between the interior and the exterior of the first basket; a disinfecting liquid; and a container configured to removably hold the liquid and the first basket when the first basket is at least partially submerged in the disinfecting liquid.

### BACKGROUND OF THE INVENTION

Children are often exposed to communicable pathogens. In order to reduce the prevalence of the spread of pathogens from an environmental object to a child and/or between children, it is critical to maintain a clean environment. Cleaning and/or disinfecting and/or sanitizing objects in the environment reduces the spread of pathogens.

### SUMMARY OF THE INVENTION

Children are often stricken with communicable diseases when placed in daycare facilities or other public settings. The inventor of the subject matter described herein has developed the systems, methods, platforms and devices to reduce and/or prevent the spread of infectious pathogens from environmental objects, for example children's toys, to children and/or between children. The systems, methods, platforms, and devices described herein are useful in environments comprising a daycare facility, a physician's offices, a dentist office, a school, a church, a public environment and any environment where children come in contact with environmental objects. In some aspects, the systems, methods, platform, and devices described herein reduce expenses to parents, for example, medical expenses, time home with sick children and other added costs caring for ill children. In some aspects, the systems, methods, platform, and devices described herein help build children's immune systems by reducing the amount of medication a child ingests in treating communicable diseases. The methods, systems, platforms and devices employ varying degrees of cleaning and/or disinfecting and/or sanitizing. In some embodiments, the systems, methods, platforms and devices described herein employ and cold sterilization.

Infants and toddlers place toys and objects in their mouth as a method of exploring their surroundings. They are often sharing and transferring mucous and saliva secretions. The inventor of the subject matter described herein, has developed the systems, methods, platforms and devices to reduce the spread of infectious pathogens through such mucous and saliva secretions. Current approaches to disinfect environmental objects employ dishwashers, and/or bleach. However, these approaches have several drawbacks, such as not all communicable pathogens are killed in a dishwasher and a bleach solution quickly loses potency due to chemical degradation and thus has to be made fresh, for example about every four hours.

In some aspects, the systems, methods, platforms and devices described herein promote development of the immune systems of young children, for example infants up to age 4, by preventing repeated illness contracted through contacting environmental object's comprising one or more communicable pathogens.

In some aspects provided herein are systems, methods, platforms and devices for disinfecting children's objects comprising: a first basket comprising one or more holes configured to allow fluid to flow between the interior and the exterior of the first basket, provided that the first basket is dimensioned such that at least one children's object is contained in the interior of the first basket; a liquid comprising glutaraldehyde, provided that the liquid is at or about room temperature; and a container configured to removably hold the liquid and the first basket when the first basket is at least partially submerged in the disinfecting liquid; provided that the at least one children's object is placed in the first basket and at least partially submerged in the liquid held in the container.

In some embodiments, the first basket comprises at least one handle. In some embodiments, the first basket comprises a lid. In further or additional embodiments, the lid is attached to the first basket or is removably attached to the first basket. In further or additional embodiments, the lid comprises at least one handle. In some embodiments, the first basket comprises at least two compartments configured to hold children's objects. In further or additional embodiments, the compartments are configured to hold different sized children's objects. In some embodiments, the first basket comprises a lid configured to cover at least one compartment. In further or additional embodiments, the lid comprises at least one handle. In further or additional embodiments, the lid is attached to the first basket or is removably attached to the first basket.

In some embodiments, the liquid further comprises one or more of cavicide, metricide, bleach, hypochlorite, chlorine dioxide, formaldehyde, hydrogen peroxide, an alcohol, an antimicrobial, an antibacterial, a germicide, an antifungal, and an antiviral. In some embodiments, the liquid temperature is at or about 20° C., at or about 21° C., at or about 22° C., at or about 23° C., at or about 24° C., at or about 25° C., at or about 26° C., at or about 27° C., at or about 28° C., at or about 29° C., or at or about 30° C. In some embodiments, the liquid temperature is less than about 20° C. or greater than about 30° C.

In some embodiments, the system further comprises a lid configured to cover the container, the first basket, or the container and the first basket. In further or additional embodiments, the lid comprises at least one handle. In further or additional embodiments, the lid is attached to the container or is removably attached to the container.

In some embodiments, the at least one children's object is a toy. In some embodiments, the at least one children's object are submerged for a time of about 90 min to about 10 hours. In some embodiments, the at least one children's object are submerged for a time of less than about 1 min, about 1 min, about 5 min, about 10 min, about 20 min, about 30 min, about 40 min, about 50 min, about 60 min, about 70 min, about 80 min, about 90 min, or greater than about 90 min. In some embodiments, the at least one children's object are submerged for a time of about 1 hour, about 2 hours, about 3 hours, about 4 hours, about 5 hours, about 6 hours, about 7 hours, about 8 hours, about 9 hours, about 10 hours, or greater than 10 hours. In some embodiments, after the at least one children's object are at least partially submerged in the disinfecting liquid, they are removed from the liquid and rinsed with water so as to remove the liquid from the surface of the at least one children's object. In some embodiments, the container comprises at least one handle. In some embodiments, the system further comprises a device to monitor the integrity of the disinfecting liquid, the device comprising one or more of a test strip, a monitor, and a sensor.

In some embodiments, the first basket is constructed from a non-porous and scratch-resistant material. In some embodiments, the first basket is constructed from one or more of polyethylene terephthalate, high-density polyethylene, vinyl ester, fiberglass, and fiberglass-reinforced plastic. In some embodiments, the first basket is constructed from a metal, an alloy, and/or a stainless steel. In some embodiments, the container is constructed from a non-porous and scratch-resistant material. In some embodiments, the container is constructed from one or more of polyethylene terephthalate, high-density polyethylene, vinyl ester, fiberglass, and fiberglass-reinforced plastic. In some embodiments, the container is constructed from a metal, an alloy, and/or a stainless steel.

In some embodiments, the systems, methods, platforms and devices described herein further comprise a plurality of baskets, provided that more than one basket is simultaneously placed in the container, and provided that the plurality of baskets is independently removed from the container. In some embodiments, the container comprises compartments, each compartment separated from another by a wall. In further or additional embodiments, each wall contains at least one hole such that liquid flows between at least two compartments. In further or additional embodiments, the first basket is dimensioned to fit in more than one compartment simultaneously. In further or additional embodiments, one or more baskets are placed in each compartment. In further or additional embodiments, the first basket comprises a plurality of grooves configured to receive a plurality of dividers so as to form a plurality of compartments in the first basket. In further or additional embodiments, one or more of the plurality of dividers comprise one or more holes so as to allow liquid to flow from one side of the divider to another side of the divider.

In some embodiments, the first basket comprises a first groove in a first interior wall and a second groove in a second interior wall, the first and second interior walls on opposite sides of the first basket. In further or additional embodiments, the first groove and the second groove are configured to receive a first divider so as to form two compartments in the first basket. In further or additional embodiments, the first basket comprises a third groove in the first interior wall and a fourth groove in the second interior wall. In further or additional embodiments, the third groove and the fourth groove are configured to receive a second divider so as to form three compartments in the first basket. In further or additional embodiments, the first basket comprises a fifth groove in a third interior wall and a sixth groove in a fourth interior wall, the third and fourth interior walls on opposite sides of the first basket. In further or additional embodiments, the fifth groove and the sixth groove are configured to receive a third divider so as to form four compartments in the first basket. In further or additional embodiments, one or more of the first and the fourth divider each comprise one or more notches so as to operably connect to each other. In further or additional embodiments, the first basket comprises a seventh groove in the third interior wall and a eighth groove in the fourth interior wall. In further or additional embodiments, the seventh groove and the eighth groove are configured to receive a fifth divider so as to form six compartments in the first basket. In further or additional embodiments, one or more of the first, second, and fourth divider comprise one or more notches so as to operably connect to each other. In further or additional embodiments, the first basket comprises a ninth groove in the third interior wall and a tenth groove in the fourth interior wall. In further or additional embodiments, the eighth groove and the ninth groove are configured to receive a sixth divider so as to form nine compartments in the first basket. In further or additional embodiments, one or more of the first, second, fourth, and sixth dividers each comprise one or more notches so as to operably connect to each other.

In further or additional embodiments, the first basket comprises a plurality of grooves in the third interior wall and a plurality of grooves in the fourth interior wall, the plurality grooves configured to receive a plurality of dividers. In further or additional embodiments, one or more of the plurality of dividers operably connected to the first and second wall comprise one or more notches so as to operably connect to one or more of the plurality of dividers operably connected to the third and fourth wall. In further or additional embodiments, one or more of the plurality of dividers operably connected to the third and fourth wall comprise one or more notches so as to operably connect to one or more of the plurality of dividers operably connected to the first and second wall. In further or additional embodiments, one or more of the plurality of dividers comprise one or more notches so as to operably connect to other dividers. In further or additional embodiments, the first basket comprises a plurality of compartments.

In some embodiments, the first basket comprises a plurality of grooves in a first interior wall and a plurality of grooves in second interior wall, the first and second interior walls on consecutive or opposite sides of the first basket. In further or additional embodiments, the plurality of grooves are configured to receive a plurality of dividers so as to form a plurality of compartments.

In some embodiments, the systems, methods, platforms and devices described herein further comprise a second basket comprising one or more holes configured to allow fluid to flow between the interior and the exterior of the basket, provided that the basket is dimensioned such that at least one children's object is contained in the interior of the basket. In further or additional embodiments, the second basket is dimensioned to fit within the first basket. In further or additional embodiments, the second basket comprises an overhang. In further or additional embodiments, the overhang is operably connected to the first basket or the container so that the bottom of the first and second basket do not touch. In further or additional embodiments, the vertical dimension of the second basket is less than the vertical dimension of the first basket. In further or additional embodiments, the second basket comprises one or more handles. In further or additional embodiments, the second basket comprises a lid. In further or additional embodiments, the lid comprises one or more handles. In further or additional embodiments, the overhang comprises the handle. In further or additional embodiments, the basket comprises a plurality of grooves configured to receive a plurality of dividers so as to form a plurality of compartments in the second basket.

In some embodiments, the first basket comprises a first slot of a first interior wall and a second slot of a second interior wall, the first and second interior walls on opposite sides of the basket. In further or additional embodiments, a first divider is inserted into the first slot and the second slot, the first divider contoured to the shape of the basket. In further or additional embodiments, the first basket comprises a third slot of a third interior wall and a fourth slot of a fourth interior wall, the third and fourth interior walls on opposite sides of the basket. In further or additional embodiments, a first divider is inserted into the first slot and the second slot, the first divider contoured to the shape of the basket, and provided that the first divider comprises a fifth slot and a sixth slot, the fifth slot and the sixth slot on opposite sides of the divider. In further or additional embodiments, second divider inserted into the third slot on the third interior wall and the fifth slot on the first divider, the second divider contoured to the shape of the basket. In further or additional embodiments, a third divider inserted into the fourth slot on the fourth interior wall and the sixth slot of the first divider, the third divider contoured to the shape of the basket.

In some embodiments, the first basket comprises a plurality of slots of a first interior wall and a plurality of slots of a second interior wall, the first and second interior walls on consecutive or opposite sides of the first basket. In further or additional embodiments, the plurality of dividers are inserted into the plurality of slots so as to form a plurality of compartments, the dividers contoured to the shape of the basket. In further or additional embodiments, the plurality of dividers comprise a plurality of slots. In further or additional embodiments, one or more additional dividers are inserted into one slot on the divider and one slot on one interior wall, the additional dividers contoured to the shape of the basket. In further or additional embodiments, one or more additional dividers are inserted into two slots on separate dividers that are inserted into slots on two interior walls of the first basket.

In some aspects provided herein are systems, methods, platforms and devices for disinfecting children's objects comprising: providing an apparatus comprising: a first basket comprising one or more holes configured to allow fluid to flow between the interior and the exterior of the first basket, provided that the first basket is dimensioned such that at least one children's object is contained in the interior of the first basket; a liquid comprising glutaraldehyde, provided that the liquid is at or about room temperature; and a container configured to removably hold the liquid and the first basket when the first basket is at least partially submerged in the disinfecting liquid; placing the at least one children's object in the first basket; at least partially submerging the first basket and the at least one children's object in the liquid held in the container for a period of time sufficient to disinfect the at least one children's object; and removing the first basket from the liquid so as to drain the liquid from the first basket.

In some embodiments, the first basket comprises at least one handle. In some embodiments, the first basket comprises a lid. In further or additional embodiments, the lid is placed on the first basket prior to at least partially submerging the first basket and the at least one children's object in the liquid held in the container. In further or additional embodiments, the lid is attached to the first basket or is removably attached to the first basket. In further or additional embodiments, the lid comprises at least one handle.

In some embodiments, the first basket comprises at least two compartments configured to hold children's objects. In further or additional embodiments, the compartments are configured to hold different sized children's objects. In further or additional embodiments, the first basket comprises a lid configured to cover at least one compartment. In further or additional embodiments, the lid is placed on the first basket prior to at least partially submerging the first basket and the at least one children's object in the liquid held in the container. In further or additional embodiments, the lid comprises at least one handle. In further or additional embodiments, the lid is attached to the first basket or is removably attached to the first basket.

In some embodiments, the liquid further comprises one or more of cavicide, metricide, bleach, hypochlorite, chlorine dioxide, formaldehyde, hydrogen peroxide, an alcohol, an antimicrobial, an antibacterial, a germicide, an antifungal, and an antiviral. In further or additional embodiments, the liquid temperature is at or about 20° C., at or about 21° C., at or about 22° C., at or about 23° C., at or about 24° C., at or about 25° C., at or about 26° C., at or about 27° C., at or about 28° C., at or about 29° C., or at or about 30° C. In further or additional embodiments, the liquid temperature is less than about 20° C. or greater than about 30° C.

In some embodiments, the apparatus further comprises a lid configured to cover the container, the first basket, or the container and the first basket. In further or additional embodiments, the lid comprises at least one handle. In further or additional embodiments, the lid is attached to the container or is removably attached to the container. In further or additional embodiments, the lid is placed on the apparatus after at least partially submerging the first basket and the at least one children's object in the liquid held in the container. In further or additional embodiments, the lid is removed from the apparatus prior to removing the first basket from the liquid so as to drain the liquid from the first basket.

In some embodiments, the at least one children's object is a toy. In some embodiments, the method further comprises submerging at least one children's object for a time of about 90 min to about 10 hours. In some embodiments, the method further comprises submerging at least one children's object for a time period of less than about 1 min, about 1 min, about 5 min, about 10 min, about 20 min, about 30 min, about 40 min, about 50 min, about 60 min, about 70 min, about 80 min, about 90 min, or greater than about 90 min. In some embodiments, the method further comprises submerging at least one children's object for a time period of about 1 hour, about 2 hours, about 3 hours, about 4 hours, about 5 hours, about 6 hours, about 7 hours, about 8 hours, about 9 hours, about 10 hours, or greater than 10 hours. In some embodiments, at least one children's object are rinsed with water so as to remove the liquid from the surface of the at least one children's object after the liquid is drained from the first basket. In some embodiments, the container comprises at least one handle. In some embodiments, the apparatus further comprises a device to monitor the integrity of the disinfecting liquid, the device comprising one or more of a test strip, a monitor, and a sensor.

In some embodiments, the first basket is constructed from a non-porous and scratch-resistant material. In some embodiments, the first basket is constructed from one or more of polyethylene terephthalate, high-density polyethylene, vinyl ester, fiberglass, and fiberglass-reinforced plastic. In some embodiments, the first basket is constructed from a metal, an alloy, and/or a stainless steel. In some embodiments, the container is constructed from a non-porous and scratch-resistant material. In some embodiments, the container is constructed from one or more of polyethylene terephthalate, high-density polyethylene, vinyl ester, fiberglass, and fiberglass-reinforced plastic. In some embodiments, the container is constructed from a metal, an alloy, and/or a stainless steel.

In some embodiments, a plurality of baskets, provided that more than one basket is simultaneously placed in the container, and provided that the plurality of baskets is independently removed from the container. In some embodiments, the container comprises compartments, each compartment separated from another by a wall. In further or additional embodiments, each wall contains at least one hole such that liquid flows between at least two compartments. In further or additional embodiments, the first basket is dimensioned to fit in more than one compartment simultaneously. In further or additional embodiments, one or more first baskets are placed in each compartment.

In some embodiments, the first basket comprises a plurality of grooves configured to receive a plurality of dividers so as to form a plurality of compartments in the first basket. In further or additional embodiments, one or more of the plurality of dividers comprise one or more holes so as to allow liquid to flow from one side of the divider to another side of the divider. In some embodiments, the first basket comprises a plurality of grooves in a first interior wall and a plurality of grooves in second interior wall, the first and second interior walls on consecutive or opposite sides of the first basket. In further or additional embodiments, the plurality of grooves are configured to receive a plurality of dividers so as to form a plurality of compartments. In further or additional embodiments, the first basket comprises a plurality of grooves in the third interior wall and a plurality of grooves in the fourth interior, the plurality grooves configured to receive a plurality of dividers. In further or additional embodiments, one or more of the plurality of dividers operably connected to the first and second wall comprise one or more notches so as to operably connect to one or more of the plurality of dividers operably connected to the third and fourth wall. In further or additional embodiments, one or more of the plurality of dividers operably connected to the third and fourth wall comprise one or more notches so as to operably connect to one or more of the plurality of dividers operably connected to the first and second wall. In further or additional embodiments, one or more of the plurality of dividers comprise one or more notches so as to operably connect to other dividers. In further or additional embodiments, the first basket comprises a plurality of compartments.

In some embodiments, the first basket comprises first groove in a first interior wall and a second groove in a second interior wall, the first and second interior walls on opposite sides of the first basket. In further or additional embodiments, the first groove and the second groove are configured to receive a first divider so as to form two compartments in the first basket. In further or additional embodiments, the first basket comprises a third groove in the first interior wall and a fourth groove in the second interior wall. In further or additional embodiments, the third groove and the fourth groove are configured to receive a second divider so as to form three compartments in the first basket. In further or additional embodiments, the first basket comprises a fifth groove in a third interior wall and a sixth groove in a fourth interior wall, the third and fourth interior walls on opposite sides of the first basket. In further or additional embodiments, the fifth groove and the sixth groove are configured to receive a third divider so as to form four compartments in the first basket. In further or additional embodiments, one or more of the first and the fourth divider each comprise one or more notches so as to operably connect to each other. In further or additional embodiments, the first basket comprises a seventh groove in the third interior wall and a eighth groove in the fourth interior wall. In further or additional embodiments, the seventh groove and the eighth groove are configured to receive a fifth divider so as to form six compartments in the first basket. In further or additional embodiments, one or more of the first, second, and fourth divider comprise one or more notches so as to operably connect to each other. In further or additional embodiments, the first basket comprises a ninth groove in the third interior wall and a tenth groove in the fourth interior wall. In further or additional embodiments, the eighth groove and the ninth groove are configured to receive a sixth divider so as to form nine compartments in the first basket. In further or additional embodiments, one or more of the first, second, fourth, and sixth dividers each comprise one or more notches so as to operably connect to each other.

In some embodiments, the systems, methods, platforms and devices described herein comprise a second basket comprising one or more holes configured to allow fluid to flow between the interior and the exterior of the basket, provided that the basket is dimensioned such that at least one children's object is contained in the interior of the basket. In further or additional embodiments, the second basket is dimensioned to fit within the first basket. In further or additional embodiments, the second basket comprises an overhang. In further or additional embodiments, the overhang is operably connected to the first basket or the container so that the bottom of the first and second basket do not touch. In further or additional embodiments, the vertical dimension of the second basket is less than the vertical dimension of the first basket. In further or additional embodiments, the second basket comprises one or more handles. In further or additional embodiments, the second basket comprises a lid. In further or additional embodiments, the lid comprises one or more handles. In further or additional embodiments, the overhang comprises the handle. In further or additional embodiments, the basket comprises a plurality of grooves configured to receive a plurality of dividers so as to form a plurality of compartments in the second basket.

In some embodiments, the method further comprises submerging at least one children's object contained in the second basket for a time of about 90 min to about 10 hours. In further or additional embodiments, the method comprises submerging at least one children's object contained in the second basket for a time period of less than about 1 min, about 1 min, about 5 min, about 10 min, about 20 min, about 30 min, about 40 min, about 50 min, about 60 min, about 70 min, about 80 min, about 90 min, or greater than about 90 min. In further or additional embodiments, the method comprises submerging at least one children's object contained in the second basket for a time period of about 1 hour, about 2 hours, about 3 hours, about 4 hours, about 5 hours, about 6 hours, about 7 hours, about 8 hours, about 9 hours, about 10 hours, or greater than 10 hours. In further or additional embodiments, at least one children's object contained in the second basket with water so as to remove the liquid from the surface of the at least one children's object after the liquid is drained from the first basket. In further or additional embodiments, the method comprises submerging at least one children's object contained in the second basket for the same or different period of time than a children's object contained in the first basket. In further or additional embodiments, the method comprises submerging at least one children's object contained in the second basket for a time period of less than about 1 min, about 1 min, about 5 min, about 10 min, about 20 min, about 30 min, about 40 min, about 50 min, about 60 min, about 70 min, about 80 min, about 90 min, or greater than about 90 min.

In some embodiments, the first basket comprises a first slot of a first interior wall and a second slot of a second interior wall, the first and second interior walls on opposite sides of the basket. In further or additional embodiments, a first divider is inserted into the first slot and the second slot, the first divider contoured to the shape of the basket. In further or additional embodiments, the first basket comprises a third slot of a third interior wall and a fourth slot of a fourth interior wall, the third and fourth interior walls on opposite sides of the basket. In further or additional embodiments, a first divider is inserted into the first slot and the second slot, the first divider contoured to the shape of the basket, and provided that the first divider comprises a fifth slot and a sixth slot, the fifth slot and the sixth slot on opposite sides of the divider. In further or additional embodiments, the system comprises a second divider inserted into the third slot on the third interior wall and the fifth slot on the first divider, the second divider contoured to the shape of the basket. In further or additional embodiments, that the system comprises a third divider inserted into the fourth slot on the fourth interior wall and the sixth slot of the first divider, the third divider contoured to the shape of the basket. In some embodiments, the first basket comprises a plurality of slots of a first interior wall and a plurality of slots of a second interior wall, the first and second interior walls on consecutive or opposite sides of the first basket. In further or additional embodiments, the plurality of dividers are inserted into the plurality of slots so as to form a plurality of compartments, the dividers contoured to the shape of the basket. In further or additional embodiments, the plurality of dividers comprise a plurality of slots. In further or additional embodiments, the system comprises one or more additional dividers, and provided that each of the one or more additional dividers are inserted into one slot on the divider and one slot on one interior wall, the additional dividers contoured to the shape of the basket. In further or additional embodiments, one or more additional dividers are inserted into two slots on separate dividers that are inserted into slots on two interior walls of the first basket.

### BRIEF DESCRIPTION OF THE DRAWINGS

FIG. 1 is an illustration of a non-limiting example of a basket.

FIG. 2 is an illustration of a non-limiting example of a basket and a divider.

FIG. 3 is an illustration of a non-limiting example of a basket and two dividers.

FIG. 4 is an illustration of a non-limiting example of a basket and three dividers.

FIG. 5 is an illustration of a non-limiting example of two dividers.

FIG. 6 is an illustration of a non-limiting example of three dividers.

FIG. 7 is an illustration of a non-limiting example of a container.

FIG. 8 is an illustration of a non-limiting example of a basket.

FIG. 9 is an illustration of a non-limiting example of a container and a basket.

FIG. 10 is an illustration of a non-limiting example of a lid.

FIG. 11 is an illustration of a non-limiting example of a lid.

FIG. 12 is an illustration of a non-limiting example of a container.

FIG. 13 is an illustration of a non-limiting example of a basket.

FIG. 14 is an illustration of a non-limiting example of a container and a basket.

FIG. 15 is an illustration of a non-limiting example of a lid.

FIG. 16 is an illustration of a non-limiting example of a lid.

### DETAILED DESCRIPTION OF THE INVENTION

### Container, Basket, Lid, and Dividers

In some aspects provided herein are systems, methods, platforms and devices to clean children's objects. In some embodiments, the systems, methods, platforms and devices described herein comprise: a first basket comprising one or more holes configured to allow fluid to flow between the interior and the exterior of the first basket, provided that the first basket is dimensioned such that at least one children's object is contained in the interior of the first basket; a liquid comprising glutaraldehyde, provided that the liquid is at or about room temperature; and a container configured to removably hold the liquid and the first basket when the first basket is at least partially submerged in the disinfecting liquid; provided that the at least one children's object is placed in the first basket and at least partially submerged in the liquid held in the container.

In some embodiments, the basket is square or rectangular. In some embodiments, the basket is cuboid. In further or additional embodiments, the basket comprises curved corners, right angular corners, obtuse angled corners, and/or acute angled corners. In some embodiments, the container is square or rectangular. In some embodiments, the container is cuboid. In further or additional embodiments, the container comprises curved corners, right angular corners, obtuse angled corners, and/or acute angled corners. In some embodiments, the basket and/or container is any vessel configured to hold solid objects and/or liquid. In further or additional embodiments, the basket is constructed such that liquid flows into and out of the interior of the basket.

In some embodiments, the container is about 12 inches wide, about 12 inches deep, and about 8.5 inches in height. In further or additional embodiments, the first basket is dimensioned to fit inside the container, for example the first basket is about 11.8 inches high, about 11.8 inches deep, and about 8.5 inches in height. In further or additional embodiments, the first basket is placed inside the container. In further or additional embodiments, when the first basket is placed inside the container, the combined height is about 9 inches. In some embodiments, the systems, methods, platforms, and devices comprise a lid. In further or additional embodiments, the lid is dimensioned to cover the first basket and/or the container. In further or additional embodiments, the lid is operatively connected to the first basket and/or the container. In some embodiments, the lid comprises a lip and/or an overhang. In some embodiments, the lid is about 12 inches wide, about 12 inches deep, and about 0.5 inches to about 3 inches in height.

In some embodiments, the container is about 12 inches wide, about 15.5 inches deep, and about 8.5 inches in height. In further or additional embodiments, the first basket is dimensioned to fit inside the container, for example the first basket is about 11.8 inches high, about 15.3 inches deep, and about 8.5 inches in height. In further or additional embodiments, the first basket is placed inside the container. In further or additional embodiments, when the first basket is placed inside the container, the combined height is about 9 inches. In some embodiments, the systems, methods, platforms, and devices comprise a lid. In further or additional embodiments, the lid is dimensioned to cover the first basket and/or the container. In further or additional embodiments, the lid is operatively connected to the first basket and/or the container. In some embodiments, the lid comprises a lip and/or an overhang. In some embodiments, the lid is about 15.5 inches wide, about 15.5 inches deep, and about 0.5 inches to about 3 inches in height.

In some embodiments, the container comprises a width of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the container comprises a depth of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the container comprises a height of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the container is dimensioned with any combination of the width, height and depth described in paragraphs [0051]-[0053].

In some embodiments, the first basket comprises a width of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the first basket comprises a depth of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the first basket comprises a height of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the first basket is dimensioned with any combination of the width, height and depth described in paragraphs [0055]-[0057].

In some embodiments, the lid comprises a width of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the lid comprises a depth of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the lid comprises a height of about 0.25 inches, about 0.5 inches, about 1 inch, about 1.5 inches, about 2 inches, about 2.5 inches, about 3 inches, about 3.5 inches, about 4 inches, about 4.5 inches, about 5 inches, and greater than about 5 inches.

In some embodiments, the lid is dimensioned with any combination of the width and depth described in paragraphs [0059]-[0061]. In some embodiments, the lid is constructed such that it is removably attached to the first basket and/or the container.

In some embodiments, the system comprises a container comprising any combination of dimensions described in paragraphs [0051]-[0053], a basket comprising any combination of dimensions described in paragraphs [0055]-[0057], and a lid comprising any combination of dimensions described in paragraphs [0059]-[0061].

In some embodiments, the first basket comprises one or more holes configured to allow fluid to flow between the interior and the exterior of the first basket, provided that the first basket is dimensioned such that at least one children's object is contained in the interior of the first basket. In some embodiments, the holes are square, rectangular, circular, elliptical, or a combination thereof. In further or additional embodiments, the first basket comprises about one hole, about two holes, about three holes, about four holes, about five holes, about six holes, about seven holes, about eight holes, about nine holes, about ten holes, about 15 holes, about 20 holes, about 25 holes, about 30 holes, about 35 holes, about 40 holes, about 45 holes, about 50 holes, about 60 holes, about 70 holes, about 80 holes, about 90 holes, about 100 holes, about 150 holes, about 200 holes, about 250 holes, about 300 holes, about 350 holes, about 400 holes, about 450 holes, about 500 holes, about 550 holes, about 600 holes, about 650 holes, about 700 holes, about 750 holes, about 800 holes, about 850 holes, about 900 holes, about 950 holes, about 1000 holes, or greater than about 1000 holes.

In some aspects the systems, methods, platforms, and devices described herein comprise a second basket comprising one or more holes configured to allow fluid to flow between the interior and the exterior of the basket, provided that the basket is dimensioned such that at least one children's object is contained in the interior of the basket. In some embodiments, the second basket is dimensioned to fit within the first basket. In some embodiments, the second basket comprises an overhang. In some embodiments, the overhang is operably connected to the first basket or the container so that the bottom of the first and second basket does not touch. In some embodiments, the vertical dimension of the second basket is less than the vertical dimension of the first basket. In some embodiments, the vertical dimension of the second basket is greater than the vertical dimension of the first basket. In some embodiments, the second basket comprises one or more handles. In some embodiments, the second basket comprises a lid. In further or additional embodiments, the lid comprises one or more handles. In further or additional embodiments, the overhang comprises the handle. In further or additional embodiments, the basket comprises a plurality of grooves configured to receive a plurality of dividers so as to form a plurality of compartments in the second basket. In further or additional embodiments, a plurality of dividers are inserted into the plurality of grooves in the second basket so as to form a plurality of compartments.

In some embodiments, the second basket comprises a depth of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the second basket comprises a height of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the second basket comprises a width of about 1 inch, about 2 inches, about 3 inches, about 4 inches, about 5 inches, about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, about 60 inches, about 61 inches, about 62 inches, about 63 inches, about 64 inches, about 65 inches, about 66 inches, about 67 inches, about 68 inches, about 69 inches, about 70 inches, about 71 inches, about 72 inches, or greater than about 72 inches.

In some embodiments, the first basket is dimensioned with any combination of the width, height and depth described in paragraphs [0066]-[0068].

In some embodiments, the second basket comprises one or more holes configured to allow fluid to flow between the interior and the exterior of the first basket, provided that the second basket is dimensioned such that at least one children's object is contained in the interior of the first basket. In some embodiments, the holes are square, rectangular, circular, elliptical, or a combination thereof. In further or additional embodiments, the first basket comprises about one hole, about two holes, about three holes, about four holes, about five holes, about six holes, about seven holes, about eight holes, about nine holes, about ten holes, about 15 holes, about 20 holes, about 25 holes, about 30 holes, about 35 holes, about 40 holes, about 45 holes, about 50 holes, about 60 holes, about 70 holes, about 80 holes, about 90 holes, about 100 holes, about 150 holes, about 200 holes, about 250 holes, about 300 holes, about 350 holes, about 400 holes, about 450 holes, about 500 holes, about 550 holes, about 600 holes, about 650 holes, about 700 holes, about 750 holes, about 800 holes, about 850 holes, about 900 holes, about 950 holes, about 1000 holes, or greater than about 1000 holes.

In some aspects of the systems, methods, devices and platforms described herein one or more of the first basket, the second basket, container, lid and dividers comprise at least one handle. In some embodiments, one or more of the first basket, the second basket, container, lid and dividers comprise two handles, three handles, four handles, or a plurality of handles. In some embodiments, the first basket, the second basket, container, lid, and dividers each comprise the same number of handles. In some embodiments, the first basket, the second basket, container, lid, and dividers each comprise a different number of handles. In some embodiments, the first basket comprises the same or different number of handles as the container. In some embodiments, the first basket comprises the same or different number of handles as the lid. In some embodiments, the first basket comprises the same or different number of handles as the dividers. In some embodiments, the container comprises the same or different number of handles as the lid. In some embodiments, the container comprises the same or different number of handles as the dividers. In some embodiments the lid comprises the same or different number of handles as the dividers. In some embodiments the first basket and the container comprise the same number of handles and the lid comprises the same or different number of handles. In some embodiments, the first basket and the lid comprise the same number of handles and the container comprises the same or different number of handles. In some embodiments, the container and the lid comprise the same number of handles and the first basket comprises the same or different number of handles. In some embodiments, the second basket comprises the same or different number of handles as the first basket, the container, the lid, and/or dividers.

In some embodiments, the one or more handles are molded into the first basket, the second basket, container, lid, and/or dividers. In some embodiments, the one or more handles are operatively connected to the first basket, the second basket, container, lid, and/or dividers. In further or additional embodiments, pins, fasteners, glue, adhesive, and/or molded fittings are used to operatively connect the one or more handles to the first basket, the second basket, container, lid and/or dividers. In further or additional embodiments, the handle is constructed from non-porous and scratch-resistant material. In some embodiments, the one or more handles are constructed from one or more of polyethylene terephthalate, high-density polyethylene, vinyl ester, fiberglass, and fiberglass-reinforced plastic. In some embodiments, the one or more handles are constructed from a metal, an alloy, and/or a stainless steel. One of skill in the art would be familiar with these materials and others that are known in the art.

In some aspects of the systems, methods, devices and platforms described herein the first basket comprises at least two compartments configured to hold children's objects. In some embodiments, the compartments are the same or different sizes. In further or additional embodiments, the compartments are configured to hold the same or different sized children's objects. In further or additional embodiments the systems, methods, devices and platforms described herein comprise a lid configured to cover at least one compartment. In further or additional embodiments, the lid comprises at least one handle. In further or additional embodiments, the lid is removably attached to the first basket so as to cover at least one compartment.

In some embodiments, a plurality of baskets are simultaneously placed in the container. In further or additional embodiments, the plurality of baskets are independently removed from the container. In some embodiments, the container comprises a plurality of compartments, each compartment separated from another by a wall. In further or additional embodiments, one or more walls comprise at least one hole such that liquid flows between at least two compartments. In further or additional embodiments, one or more walls comprise a plurality of holes such that liquid flows between at least two compartments. In some embodiments, the first basket is dimensioned to fit in more than one compartment simultaneously. In further or additional embodiments, one or more baskets are placed in each compartment.

In some aspects of the systems, methods, platforms, and devices described herein, the first basket comprises a plurality of grooves configured to receive a plurality of dividers so as to form a plurality of compartments in the first basket. In some embodiments, the grooves are molded into at least one inside wall of the first basket. In some embodiments, the grooves are recessed from the surface of the wall. In some embodiments, the grooves are raised from the surface of the inside wall. In some embodiments, one or more of the plurality of dividers comprise one or more holes so as to allow liquid to flow from one side of the divider to another side of the divider.

In some embodiments, the first basket comprises a first groove in a first interior wall and a second groove in a second interior wall, the first and second interior walls on opposite sides of the first basket. In some embodiments, the grooves are recessed from the surface of the wall. In some embodiments, the grooves are raised from the surface of the inside wall. In further or additional embodiments, first groove and the second groove are configured to receive a first divider so as to form two compartments in the first basket. In further or additional embodiments, a first divider is inserted into the first and second grooves so as to form two compartments in the first basket. In further or additional embodiments, the first basket comprises a third groove in the first interior wall and a fourth groove in the second interior wall. In further or additional embodiments, the third groove and the fourth groove are configured to receive a second divider so as to form three compartments in the first basket. In further or additional embodiments, a second divider is inserted into the third and fourth grooves so as to form three compartments in the first basket. In some embodiments, the first basket comprises a fifth groove in a third interior wall and a sixth groove in a fourth interior wall, the third and fourth interior walls on opposite sides of the first basket. In further or additional embodiments, the fifth groove and the sixth groove are configured to receive a third divider so as to form four compartments in the first basket. In further or additional embodiments, a third divider is inserted into the fifth groove and the sixth groove so as to form four compartments. In further or additional embodiments, one or more of the first and the fourth divider each comprise one or more notches so as to operably connect to each other. In some embodiments, the first basket comprises a seventh groove in the third interior wall and a eighth groove in the fourth interior wall. In further or additional embodiments, the seventh groove and the eighth groove are configured to receive a fifth divider so as to form six compartments in the first basket. In further or additional embodiments, a fifth divider is inserted into the seventh groove and the eighth groove so as to form six compartments. In further or additional embodiments, one or more of the first, second, and fourth divider comprise one or more notches so as to operably connect to each other. In some embodiments, the first basket comprises a ninth groove in the third interior wall and a tenth groove in the fourth interior wall. In further or additional embodiments, the eighth groove and the ninth groove are configured to receive a sixth divider so as to form nine compartments in the first basket. In further or additional embodiments, a sixth divider is inserted into the eighth groove and the ninth groove so as to form nine compartments. In further or additional embodiments, one or more of the first, second, fourth, and sixth dividers each comprise one or more notches so as to operably connect to each other.

In some embodiments, the first basket comprises a plurality of grooves in a first interior wall and a plurality of grooves in second interior wall, the first and second interior walls on consecutive or opposite sides of the first basket. In further or additional embodiments, the plurality of grooves are configured to receive a plurality of dividers so as to form a plurality of compartments. In further or additional embodiments a plurality of dividers are inserted into the plurality of grooves so as to form a plurality of compartments. In some embodiments, the first basket comprises a plurality of grooves in the third interior wall and a plurality of grooves in the fourth interior, the plurality grooves configured to receive a plurality of dividers. In further or additional embodiments, a plurality of dividers are inserted into the plurality of grooves in the third interior wall and the plurality of grooves in the fourth interior wall so as to form a plurality of compartments. In further or additional embodiments, one or more of the plurality of dividers operably connected to the first and second wall comprise one or more notches so as to operably connect to one or more of the plurality of dividers operably connected to the third and fourth wall. In further or additional embodiments, one or more of the plurality of dividers operably connected to the third and fourth wall comprise one or more notches so as to operably connect to one or more of the plurality of dividers operably connected to the first and second wall. In further or additional embodiments, one or more of the plurality of dividers comprise one or more notches so as to operably connect to other dividers.

In some aspects of the systems, methods, devices and platforms described herein, the first basket comprises a first slot of a first interior wall and a second slot of a second interior wall, the first and second interior walls on opposite sides of the basket. In some embodiments, a first divider is inserted into the first slot and the second slot, the first divider contoured to the shape of the basket. In further or additional embodiments, the first basket comprises a third slot of a third interior wall and a fourth slot of a fourth interior wall, the third and fourth interior walls on opposite sides of the basket. In further or additional embodiments, a first divider is inserted into the first slot and the second slot, the first divider contoured to the shape of the basket, and provided that the first divider comprises a fifth slot and a sixth slot, the fifth slot and the sixth slot on opposite sides of the divider. In further or additional embodiments, a second divider is inserted into the third slot on the third interior wall and the fifth slot on the first divider, the second divider contoured to the shape of the basket. In further or additional embodiments, a third divider inserted into the fourth slot on the fourth interior wall and the sixth slot of the first divider, the third divider contoured to the shape of the basket.

In some embodiments, the first basket comprises a plurality of slots of a first interior wall and a plurality of slots of a second interior wall, the first and second interior walls on consecutive or opposite sides of the first basket. In further or additional embodiments, the plurality of dividers are inserted into the plurality of slots so as to form a plurality of compartments, the dividers contoured to the shape of the basket. In further or additional embodiments, the plurality of dividers comprise a plurality of slots. In further or additional embodiments, one or more additional dividers, and provided that each of the one or more additional dividers are inserted into one slot on the divider and one slot on one interior wall, the additional dividers contoured to the shape of the basket.

In some aspects of the systems, methods, devices and platforms described herein one or more of the first basket, container, lid and dividers are constructed from a non-porous and scratch-resistant material. In some embodiments, one or more of the first basket, container, lid and dividers are constructed from one or more of polyethylene terephthalate, high-density polyethylene, vinyl ester, fiberglass, and fiberglass-reinforced plastic. In some embodiments, one or more of the first basket, container, lid and dividers are constructed from a metal, an alloy, and/or a stainless steel. One of skill in the art would be familiar with these materials and others that are known in the art.

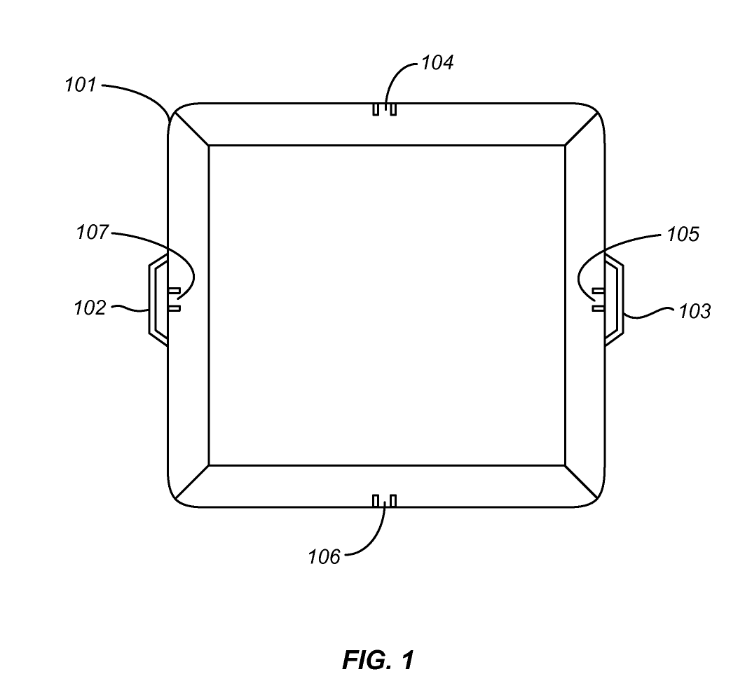

An illustration of a non-limiting example of the systems, methods, platforms and devices described herein depicted in FIG. 1 comprises a basket 101 comprising a first handle 102, a second handle 103, a first slot 104, a second slot 105, a third slot 106, and a fourth slot 107. In some embodiments, the first, second, third and fourth slots are grooves.

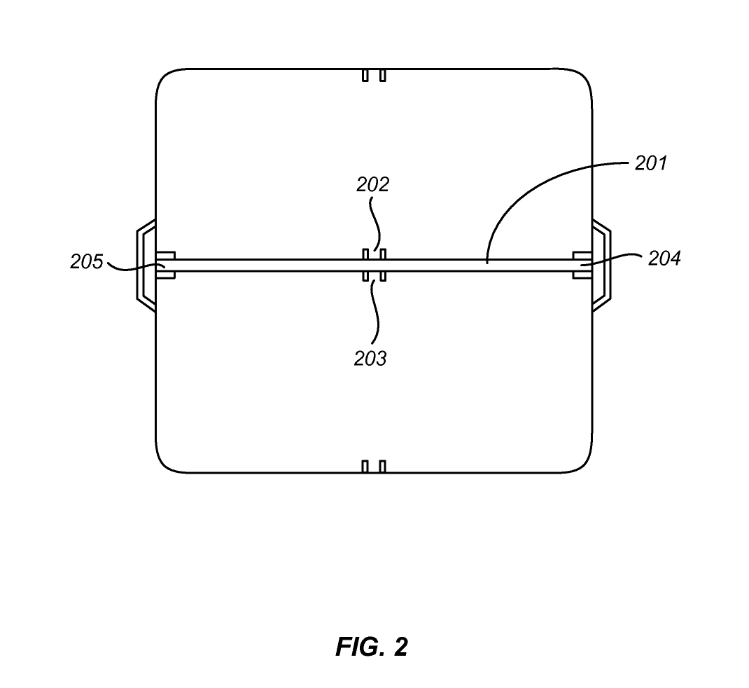

An illustration of a non-limiting example of the systems, methods, platforms and devices described herein depicted in FIG. 2 comprises a basket and a divider 201 comprising a first slot 202 and a second slot 203, the divider inserted into a third slot 204 and a fourth slot 205 of the basket. In some embodiments, one or more of the first, second, third and fourth slots are grooves.

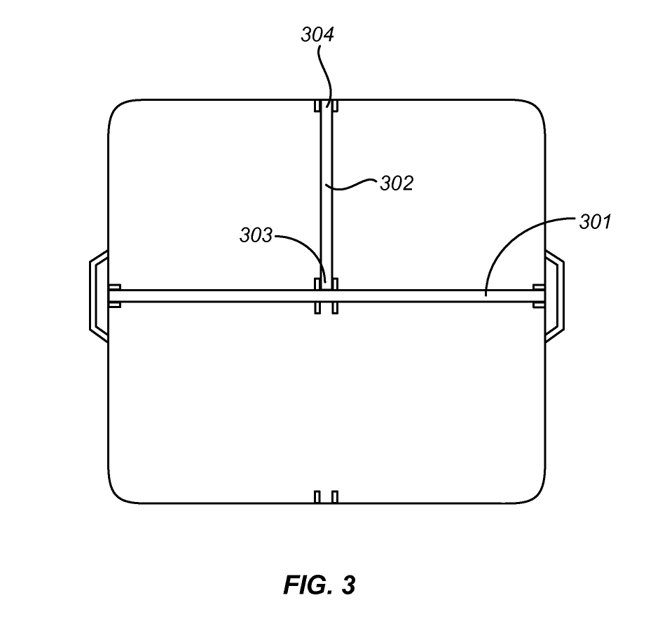

An illustration of a non-limiting example of the systems, methods, platforms and devices described herein depicted in FIG. 3 comprises a basket, a first divider 301 and a second divider 302, the second divider inserted into a first slot 303 of the first divider and a second slot 304 of the basket. In some embodiments, one or more of the first and second slots are grooves.

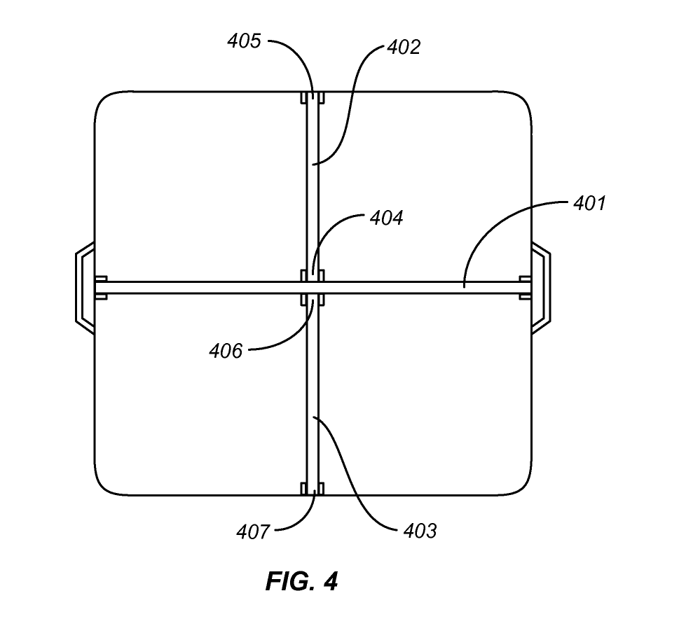

An illustration of a non-limiting example of the systems, methods, platforms and devices described herein depicted in FIG. 4 comprises a basket, a first divider 401, a second divider 402, and a third divider 403, the second divider inserted into a first slot 404 of the first divider and a second slot 405 of the basket and the third divider inserted into a third slot 406 of the first divider and a fourth slot 407 of the basket. In some embodiments, one or more of the first, second, third and fourth slots are grooves.

An illustration of a non-limiting example of the systems, methods, platforms and devices described herein depicted in FIG. 5 comprises a second divider and a third divider. The upper panel 501 depicts a non-limiting example of a side view of the second and third divider inserted into the slots or grooves of another divider. The lower panel 502 depicts a non-limiting example of a side view of the second and third dividers.

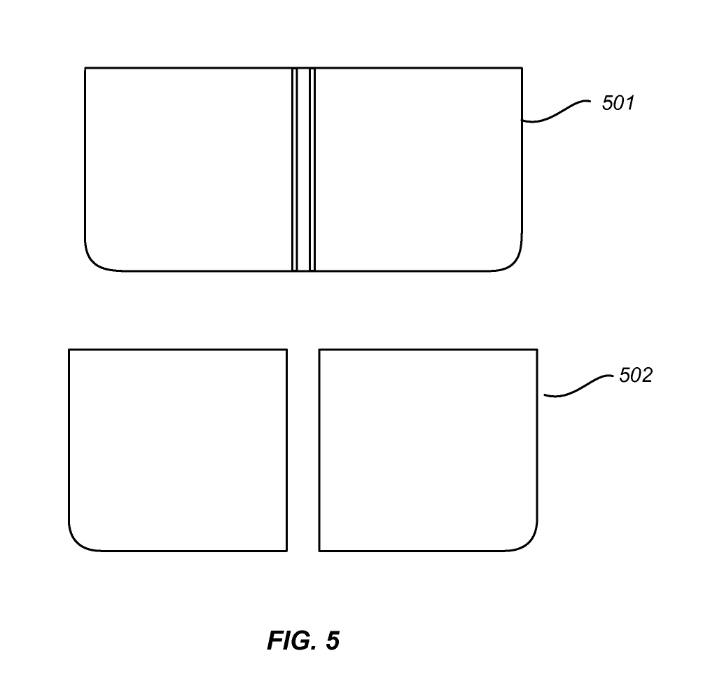

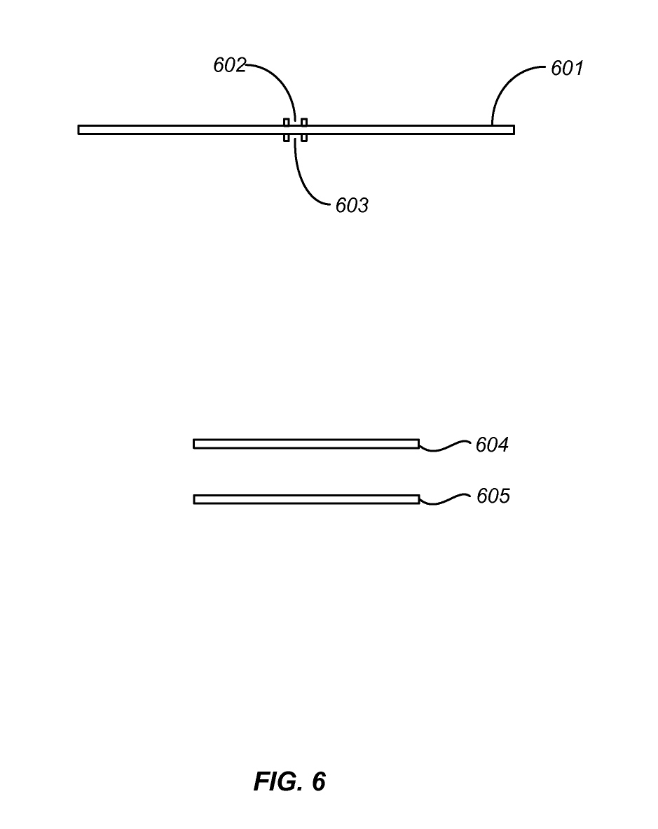

An illustration of a non-limiting example of the systems, methods, platforms and devices described herein depicted in FIG. 6 comprises a first divider 601, second divider 604, and a third divider 605, the first divider comprising a first slot 602 and a second slot 603. In some embodiments, one or more of the first and second slots are grooves.

An illustration of a non-limiting example of the systems, methods, platforms and devices described herein depicted in FIG. 7 comprises a container comprising a first handle 701 and a second handle 702.

An illustration of a non-limiting example of the systems, methods, platforms and devices described herein depicted in FIG. 8 comprises a basket comprising a first handle 802 and a second handle 803. In some embodiments, the basket comprises a plurality of holes 801.

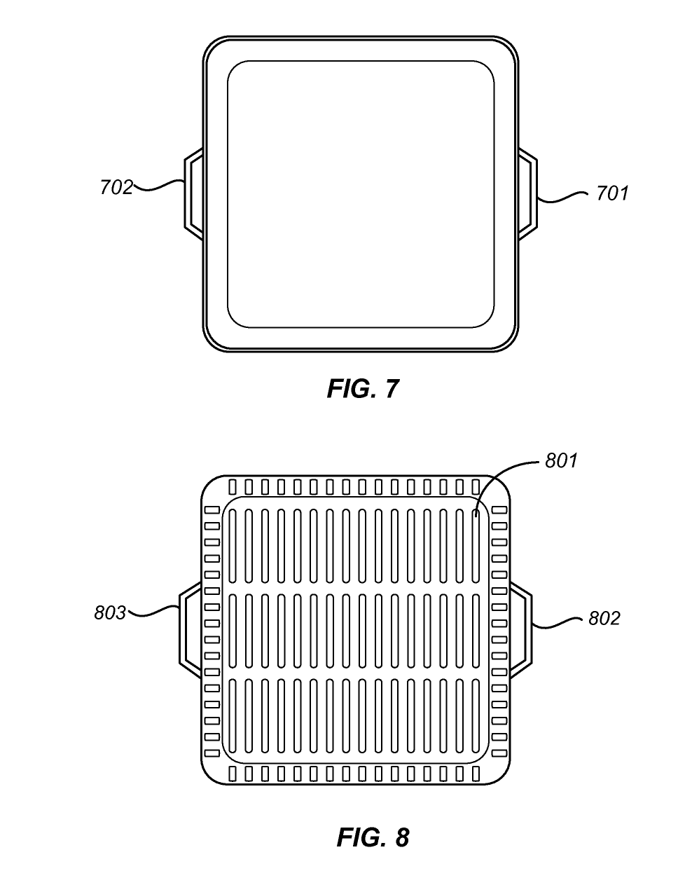

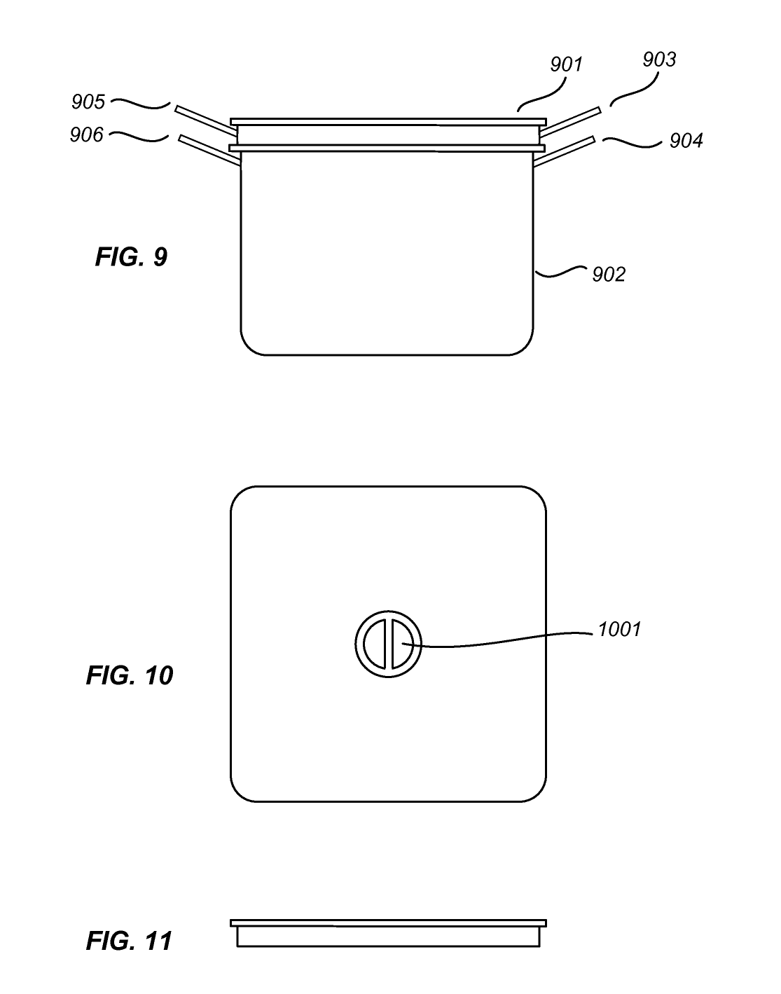

An illustration of a non-limiting example of the systems, methods, platforms and devices described herein depicted in FIG. 9 comprises a container 902, a basket 901 inserted into the container, a first handle 903 of the basket, a second handle 905 of the basket, a third handle 904 of the container, and a fourth handle 906 of the container. In some embodiments, the handles of the basket and the container are spaced such that the basket can be easily removed from the container using the handles of the basket.

An illustration of a non-limiting example of the systems, methods, platforms and devices described herein depicted in FIG. 10 comprises a lid comprising a handle 1001. An illustration of a non-limiting example of a side view of the lid is depicted in FIG. 11.

An illustration of a non-limiting example of the systems, methods, platforms and devices described herein depicted in FIG. 12 comprises a container comprising a first handle 1201 and a second handle 1202.

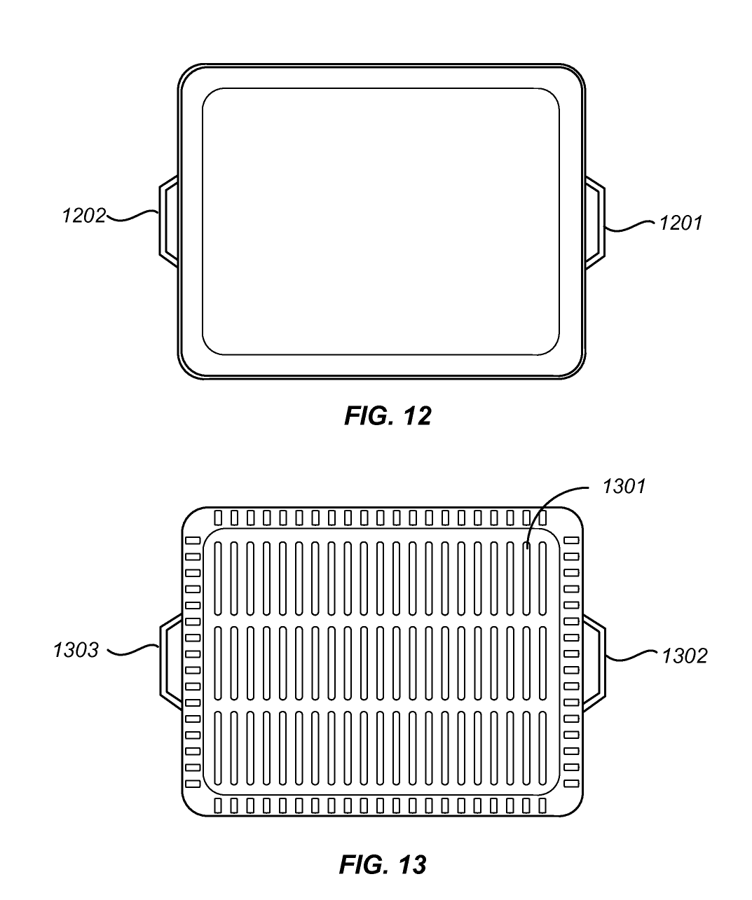

An illustration of a non-limiting example of the systems, methods, platforms and devices described herein depicted in FIG. 13 comprises a basket comprising a first handle 1302 and a second handle 1303. In some embodiments, the basket comprises a plurality of holes 1301.

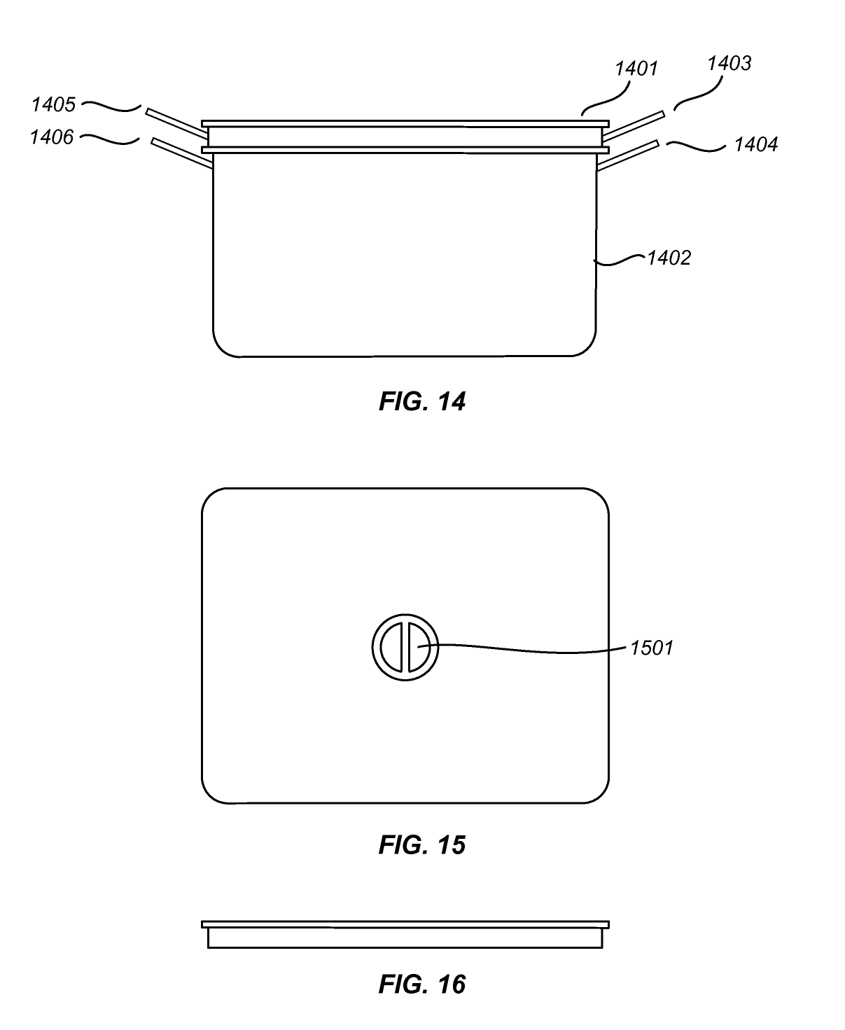

An illustration of a non-limiting example of the systems, methods, platforms and devices described herein depicted in FIG. 14 comprises a container 1402, a basket 1401 inserted into the container, a first handle 1403 of the basket, a second handle 1405 of the basket, a third handle 1404 of the container, and a fourth handle 1406 of the container. In some embodiments, the handles of the basket and the container are spaced such that the basket can be easily removed from the container using the handles of the basket.

An illustration of a non-limiting example of the systems, methods, platforms and devices described herein depicted in FIG. 15 comprises a lid comprising a handle 1501. An illustration of a non-limiting example of a side view of the lid is depicted in FIG. 16.

### Solutions/Chemicals

In some aspects the systems, methods, devices and platforms described herein comprise a cleaning and/or disinfecting and/or sanitizing liquid. In some embodiments the liquid comprises one or more of glutaraldehyde, cavicide, metricide, bleach, hypochlorite, chlorine dioxide, formaldehyde, hydrogen peroxide, an alcohol, an antimicrobial, an antibacterial, a germicide, an antifungal, and an antiviral. In some embodiments the cleaning and/or disinfecting and/or sanitizing liquid comprises glutaraldehyde. In some embodiments, the integrity of the cleaning and/or disinfecting and/or sanitizing liquid is tested using test strips configured to detect the cleaning and/or disinfecting and/or sanitizing chemical(s) in the liquid.

In some embodiments, the cleaning and/or disinfecting and/or sanitizing liquid is stable at a temperature, at or about 10° C., at or about 11° C., at or about 12° C., at or about 13° C., at or about 14° C., at or about 15° C., at or about 16° C., at or about 17° C., at or about 18° C., at or about 19° C., at or about 20° C., at or about 21° C., at or about 22° C., at or about 23° C., at or about 24° C., at or about 25° C., at or about 26° C., at or about 27° C., at or about 28° C., at or about 29° C., or at or about 30° C., at or about 31° C., at or about 32° C., at or about 33° C., at or about 34° C., at or about 35° C., at or about 36° C., at or about 37° C., at or about 38° C., at or about 39° C., and/or at or about 40° C. In some embodiments, the cleaning and/or disinfecting and/or sanitizing liquid is stable at a temperature less than about 10° C. or greater than about 40° C.

In some embodiments, the cleaning and/or disinfecting and/or sanitizing liquid is stable for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1 month, about 2 months, about 3 months, about 4 months, about 5 months, about 6 months, about 7 months, about 8 months, about 9 months, about 10 months, about 11 months, and/or about 12 months.

### Cleaning/Disinfecting/Sanitizing Protocols

In some aspects of the systems, methods, platforms and devices described herein, one or more objects, for example children's objects, are placed inside the first basket and at least partially submerged in a cleaning and/or disinfecting and/or sanitizing liquid contained in the container. In some embodiments, one or more objects are at least partially submerged for about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 75 minutes, about 90 minutes, about 105 minutes, about 120 minutes, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1.5 days, about 2 days, about 2.5 days, about 3 days, about 3.5 days, about 4 days, about 4.5 days, about 5 days, about 5.5 days, about 6 days, about 6.5 days, and/or about 7 days. In some embodiments, one or more objects are at least partially submerged for greater than about 7 days. In some embodiments, the one or more objects are at least partially submerged in the liquid at a temperature at or about 10° C., at or about 11° C., at or about 12° C., at or about 13° C., at or about 14° C., at or about 15° C., at or about 16° C., at or about 17° C., at or about 18° C., at or about 19° C., at or about 20° C., at or about 21° C., at or about 22° C., at or about 23° C., at or about 24° C., at or about 25° C., at or about 26° C., at or about 27° C., at or about 28° C., at or about 29° C., or at or about 30° C., at or about 31° C., at or about 32° C., at or about 33° C., at or about 34° C., at or about 35° C., at or about 36° C., at or about 37° C., at or about 38° C., at or about 39° C., and/or at or about 40° C. In some embodiments, the one or more objects are at least partially submerged in the liquid at a temperature less than about 10° C. or greater than about 40° C.

In some embodiments, the time period in which an object is at least partially submerged in a cleaning and/or disinfecting and/or sanitizing liquid is varied defending on whether a user is cleaning, disinfecting, or sanitizing the object. In further or additional embodiments, the longer the period of time in which an object is at least partially submerged in the cleaning and/or disinfecting and/or sanitizing liquid the more disinfected or sterile the object becomes. In some embodiments, the temperature at which an object is at least partially submerged in a cleaning and/or disinfecting and/or sanitizing liquid is varied defending on whether a user is cleaning, disinfecting, or sanitizing the object. In further or additional embodiments, the colder the temperature in which an object is at least partially submerged in the cleaning and/or disinfecting and/or sanitizing liquid the less disinfected or sterile the object becomes.

In some embodiments, after one or more objects are at least partially submerged, the first basket is lifted out of the solution so as to remove the one or more objects from the liquid. In further or additional embodiments, the basket comprising the one or more objects is transferred to a sink and the one or more objects are rinsed with water and dried.

In some aspects of the systems, methods, platforms, and devices described herein one or more objects, for example children's objects, are placed in the first basket and the first basket comprising the one or more objects is placed in the container. In some embodiments, a cleaning and/or disinfecting and/or sanitizing liquid, for example cavicide, is applied to the one or more objects, for example using a spray bottle. In some embodiments, the cleaning and/or disinfecting and/or sanitizing liquid contacts the surface of the one or more objects such that pathogens on the objects are no longer virulent, for example, the cleaning and/or disinfecting and/or sanitizing liquid contacts the one or more object's surface for 1-5 minutes. In further or additional embodiments, after the liquid contacts the surface of the one or more objects for a period of time, the first basket comprising the one or more objects is lifted from the container and transferred to a sink and the one or more objects are rinsed with water and dried. In some embodiments, transfer of the first basket to the sink is convenient for a user. In further or additional embodiments, the first basket is conveniently transferred to the sink and placed under running water so as to rinse the at least one children's object. In further or additional embodiments, the convenience of using the systems, methods, platforms, and devices described promotes usage to clean and/or disinfect and/or sanitize the at least one children's object.

In some embodiments, the methods described herein are combined with additional safety precautions. In some embodiments, a sick child is not allowed to attend school and/or daycare until a child has been cleared of any fever or illness for at least 24 hours. In some embodiments, if a child becomes sick while in daycare and/or school, the child is isolated and placed in a designated area until released to parent and/or guardian. In some embodiments, all objects that were in contact with the sick child undergo a cleaning process, for example by contacting the objects with the cleaning and/or disinfecting and/or sanitizing liquid for a period of time, for example 10 hours. In further or additional embodiments, the objects are repeatedly cleaned, for example repeatedly cleaned for about 1 week.

In some embodiments, when an outbreak of a pathogen is detected in an environment, one or more objects evidenced to have contacted the pathogen and/or suspected to have contacted the pathogen are cleaned and/or disinfected and/or sanitized by contacting the objects with the cleaning and/or disinfecting and/or sanitizing liquid for a period of time, for example 10 hours. In further or additional embodiments, the objects are repeatedly cleaned, for example repeatedly cleaned for about 1 week.

### Children's Objects and Furniture

In some aspects of the systems, methods, platforms and devices described herein, the one or more objects comprise a children's toy, plaything, a trinket, a device, an instrument, a tool, or any object that comes in contact with a child. In some embodiments, children's objects are fabricated using plastic and/or a non-porous material resistant to a cleaning and/or disinfecting and/or sanitizing liquid, for example glutaraldehyde. In further or additional embodiments, the at least one children's objects fabricated using plastic and/or a non-porous material resistant to a cleaning and/or disinfecting and/or sanitizing liquid are educational toys. In some embodiments, furniture surfaces that children commonly contact are manufactured using plastic and/or a non-porous material resistant to a cleaning and/or disinfecting and/or sanitizing liquid, for example glutaraldehyde.

In some aspects of the systems, methods, platforms and devices described herein the one or more objects comprises any object that will at least partially fit in the basket. In some embodiments, the one or more objects comprise a medical instrument, a dental instrument, a surgical instrument, a physician's instrument, and a combination thereof. In some embodiments, the one or more objects comprises a dental instrument, for example a mouth mirror, a hand-held probe, a sickle probe, a contra-angled probe, a periodontal probe, a briault probe, college tweezers, a saliva ejector, a syringe, a needle, a rubber dam, a dental bur, any dental instrument, and a combination thereof. In some embodiments, the one or more objects comprises a dental instrument, for example a heat sensitive instrument made of resin and/or plastic. In further or additional embodiments, the one or more objects comprises a dental instrument, for example a plastic suction tip, a composite brush handle, an impression tray, a cheek retractor, and a combination thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for disinfecting children's objects comprising:
a. a first basket comprising one or more holes configured to allow fluid to flow between an interior and an exterior of the first basket;
b. a liquid comprising glutaraldehyde, provided that the liquid is at or about room temperature;
c. a container configured to hold the liquid and the first basket such that the liquid enters the first basket when the first basket is at least partially submerged in the liquid; and
d) a children's object comprising a toy, a trinket, or a plaything, provided that:
(i) the children's object is placed in the first basket and at least partially submerged in the liquid held in the container; and
(ii) the children's object is not a medical instrument, a dental instrument, a surgical instrument, or a physician's instrument.

2. The system of claim 1, provided that the first basket comprises at least two compartments configured to hold children's objects, the children's objects comprising a toy, a trinket, or a plaything, provided that the children's objects are not medical instruments, dental instruments, surgical instruments, or physician's instruments.

3. The system of claim 2, provided that the compartments are configured to hold different sized children's objects, the children's objects comprising a toy, a trinket, or a plaything, provided that the children's objects are not medical instruments, dental instruments, surgical instruments, or physician's instruments.

4. The system of claim 1, provided that the liquid further comprises one or more of cavicide, metricide, bleach, hypochlorite, chlorine dioxide, formaldehyde, hydrogen peroxide, an alcohol, an antimicrobial, an antibacterial, a germicide, an antifungal, and an antiviral.

5. The system of claim 1, comprising a plurality of baskets, provided that more than one basket is simultaneously placed in the container, and provided that each of the plurality of baskets is independently removable from the container.

6. The system of claim 1, provided that the first basket comprises a first slot of a first interior wall and a second slot of a second interior wall, the first and second interior walls on opposite sides of the basket.

7. The system of claim 6, provided that a first divider is inserted into the first slot and the second slot, the first divider contoured to the shape of the basket.

8. The system of claim 6, provided that the first basket comprises a third slot of a third interior wall and a fourth slot of a fourth interior wall, the third and fourth interior walls on opposite sides of the basket.

9. The system of claim 8, provided that a first divider is inserted into the first slot and the second slot, the first divider contoured to the shape of the basket, and provided that the first divider comprises a fifth slot and a sixth slot, the fifth slot and the sixth slot on opposite sides of the divider.

10. The system of claim 9, provided that the system comprises a second divider inserted into the third slot on the third interior wall and the fifth slot on the first divider, the second divider contoured to the shape of the basket.

11. The system of claim 10, provided that the system comprises a third divider inserted into the fourth slot on the fourth interior wall and the sixth slot of the first divider, the third divider contoured to the shape of the basket.

12. A method for disinfecting children's objects comprising:
a. providing an apparatus comprising:
i) a first basket comprising one or more holes configured to allow fluid to flow between an interior and an exterior of the first basket;
ii) a liquid comprising glutaraldehyde, provided that the liquid is at or about room temperature;
iii) a container configured to hold the liquid and the first basket such that the liquid enters the first basket when the first basket is at least partially submerged in the liquid; and
iv) a children's object comprising a toy, a trinket, or a plaything, provided that the children's object is not a medical instrument, a dental instrument, a surgical instrument, or a physician's instrument;
b. placing the children's object in the first basket;
c. at least partially submerging the first basket into the container such that the children's object is at least partially submerged in the liquid held in the container for a period of time sufficient to disinfect at least a portion of the children's object; and
d. removing the first basket from the liquid so as to drain the liquid from the first basket.

13. The method of claim 12, provided that the liquid further comprises one or more of cavicide, metricide, bleach, hypochlorite, chlorine dioxide, formaldehyde, hydrogen peroxide, an alcohol, an antimicrobial, an antibacterial, a germicide, an antifungal, and an antiviral.

14. The method of claim 12, provided that the liquid temperature is less than about 20° C., at or about 20° C., at or about 21° C., at or about 22° C., at or about 23° C., at or about 24° C., at or about 25° C., at or about 26° C., at or about 27° C., at or about 28° C., at or about 29° C., at or about 30° C., or greater than about 30° C.

15. The method of claim 12, further comprising submerging the children's object for a time period of less than about 1 min, about 1 min, about 5 min, about 10 min, about 20 min, about 30 min, about 40 min, about 50 min, about 60 min, about 70 min, about 80 min, about 90 min, about 2 hours, about 3 hours, about 4 hours, about 5 hours, about 6 hours, about 7 hours, about 8 hours, about 9 hours, about 10 hours, or greater than 10 hours.

16. The method of claim 12, further comprising rinsing the children's object with water so as to remove the liquid from the surface of the children's object after the liquid is drained from the first basket.

17. The method of claim 12, provided that the apparatus further comprises a device to monitor the integrity of the disinfecting liquid, the device comprising one or more of a test strip, a monitor, and a sensor.

18. The method of claim 12, comprising a second basket comprising one or more holes configured to allow liquid to flow between the interior and the exterior of the second basket, provided that the second basket is dimensioned such that the children's object is contained in the interior of the second basket.

19. The method of claim 18, further comprising submerging the children's object contained in the second basket for a time period of less than about 1 min, about 1 min, about 5 min, about 10 min, about 20 min, about 30 min, about 40 min, about 50 min, about 60 min, about 70 min, about 80 min, about 90 min, about 2 hours, about 3 hours, about 4 hours, about 5 hours, about 6 hours, about 7 hours, about 8 hours, about 9 hours, about 10 hours, or greater than 10 hours.

20. The method of claim 18, further comprising rinsing the children's object contained in the second basket with water so as to remove the liquid from the surface of the children's object after the liquid is drained from the first second basket.

21. The system of claim 1, comprising a device to monitor the integrity of the disinfecting liquid, the device comprising one or more of a test strip, a monitor, and a sensor.

22. The system of claim 1, comprising a second basket comprising one or more holes configured to allow fluid to flow between the interior to the exterior of the second basket, provided that the second basket is dimensioned such that the children's object is contained in the interior of the second basket.

23. The system of claim 22, provided that the second basket is dimensioned to fit within the first basket.

24. The system of claim 1, provided that the first basket comprises a first groove in a first interior wall and a second groove in a second interior wall, the first and second interior walls on opposite sides of the first basket.

25. The system of claim 24, provided that the first groove and the second groove are configured to receive a first divider so as to form two compartments in the first basket.

26. The system of claim 25, provided that the first basket comprises a third groove in a third interior wall and a fourth groove in a fourth interior wall, the third and fourth interior walls on opposite sides of the first basket.

27. The system of claim 26, provided that the third groove and the fourth groove are configured to receive a second divider so as to form four compartments in the first basket.

28. The system of claim 27, provided that one or more of the first and the second divider comprise one or more notches so as to operably connect to each other.

29. The system of claim 1, provided that the first basket comprises a plurality of grooves in a first interior wall and a plurality of grooves in a second interior wall, the first and second interior walls on consecutive or opposite sides of the first basket.

30. The system of claim 29, provided that the plurality of grooves are configured to receive a plurality of dividers so as to form a plurality of compartments.
